(19) United States
(12) Patent Application Publication
(10) Pub. No.: US 2024/0173030 A1
(43) Pub. Date: May 30, 2024

(54) ANCHOR DEVICES, SYSTEMS, AND METHODS FOR NON-ADHERENT ANATOMICAL STRUCTURES

(71) Applicant: BOSTON SCIENTIFIC SCIMED, INC., MAPLE GROVE, MN (US)

(72) Inventors: Peter L. Dayton, Brookline, MA (US); Paul Smith, Smithfield, RI (US); Douglas Melanson, Natick, MA (US)

(73) Assignee: BOSTON SCIENTIFIC SCIMED, INC., MAPLE GROVE, MN (US)

(21) Appl. No.: 18/523,004

(22) Filed: Nov. 29, 2023

Related U.S. Application Data
(60) Provisional application No. 63/428,801, filed on Nov. 30, 2022.

(51) Int. Cl. A61B 17/11

(52) U.S. Cl. CPC A61B 17/11; A61B 2017/00867

(57) ABSTRACT

An anchor capable of being delivered in a delivery configuration and deployed through anatomical tissue with the use of a delivery device. Upon deployment, the anchor shifts into a deployment configuration with at least a distal anchor portion extending transverse to a medial portion of the anchor. The anchor is removable from the anatomical tissue with the use of a retrieval device which grasps a proximal end of the anchor and proximally retracts the anchor into a flexible tubular element to return the anchor to the delivery configuration.

### CROSS-REFERENCE TO RELATED APPLICATIONS

This application claims the benefit of priority under 35 U.S.C. § 119 to U.S. Provisional Application No. 63/428,801 filed Nov. 30, 2022, the entire disclosure of which is hereby incorporated by reference herein for all purposes.

### FIELD

The present disclosure relates generally to the field of medical anchoring devices, systems, and methods. In some aspects, the present disclosure relates to devices, systems, and methods for holding anatomical structures in apposition with each other. The present disclosure further relates to medical anchoring devices, systems, and methods capable of removal from a deployment site.

### BACKGROUND

Complex endoscopic ultrasound (EUS) guided procedures (such as in the gastrointestinal tract) require adequate and accurate positioning while maintaining clear ultrasound imaging in order to perform a procedure safely and effectively to the desired access site. Such procedures may include various tasks, including, without limitation, cutting, cauterizing, delivering a treatment device, etc., which typically require adequate and accurate positioning and control. Although endoscopic imaging modalities, such as fluoroscopy and endoscopic ultrasound (EUS), have the ability to view anatomical structures beyond the proximate, visually accessible, tissue in front of the endoscope, therapeutic endoscopic procedures may be limited by the difficulty of controlling tissue beyond the tissue plane visually accessible to the endoscope. Different anatomical sites pose unique challenges, such as with positioning, distance, and shape. For example, placing a stent between two non-adherent anatomical structures (e.g., tissue walls) such as in a gastrojejunostomy, hepaticogastrostomy, or gallbladder drainage into either the stomach or duodenum is technically challenging due to the lack of tools to visualize, stabilize, and, in some cases, inflate the target site. For instance, in certain procedural situations, it is challenging to place a stent in proper alignment with non-adherent anatomical structures (e.g., tissue walls). For instance, advancement of a stent between two non-adherent anatomical structures may push the distal non-adherent anatomical structure away from the proximal non-adherent anatomical structure. In some situations, the stent may not be able to securely hold the non-adherent structures in apposition as desired, or necessary, for the procedure. Moreover, once a site is accessed, physicians need to be able to maintain the access without the proximal or distal structure moving away from or slipping with respect to the proximal structure, which may result in losing the access site. In some instances, the distal site cannot readily be re-accessed if it moves away from the proximal structure, leaving the patient with a perforation in the distal structure and potentially harmful leakage of bodily fluid therefrom. All of these challenges can lead to a failed procedure with serious complications in a patient population already in extremely poor health where there is no room for failure. Improvement to delivery and deployment of devices which move anatomical structures into apposition and/or hold anatomical structures into apposition would be welcome in the field.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, an anchor capable of extending through anatomical tissue at a deployment site includes a distal end forming a distal anchor portion; a proximal end forming a proximal anchor portion; and a medial portion, between the distal end and the proximal end, configured to extend through anatomical tissue. In some aspects, the anchor is shiftable between an elongated delivery configuration and a deployed configuration; at least the distal anchor portion is formed when the anchor shifts into the deployed configuration by bending into a configuration extending transverse to the medial portion; and a free end of the proximal anchor portion extends away from the deployment site to facilitate access thereto for removal of the anchor from the deployment site.

Optionally, at least one of the distal anchor portion or the proximal anchor portion is curved. Optionally, the distal anchor portion is curved in a direction transverse to and away from the medial portion. Additionally or alternatively, the distal anchor portion has a free end directed away from the medial portion. Additionally or alternatively, the distal anchor portion has a free end extending along the anatomical tissue. Additionally or alternatively, the distal anchor portion has a free end embedded into the anatomical tissue. Additionally or alternatively, the proximal anchor portion is curved in a direction transverse to an away from the medial portion. Additionally or alternatively, the proximal anchor portion has a free end extending away from the medial portion to be graspable to remove the anchor from the deployment site. Additionally or alternatively, the anchor is shiftable from the deployed configuration to the delivery configuration upon being pulled into a lumen of a tubular element for removal from the deployment site.

Optionally, the anchor is formed from a shape memory material. Optionally, the anchor is formed from a wire.

In some embodiments, the medial portion comprises a suture. Optionally, the anchor is removable from the deployment site upon cutting the suture.

In accordance with various principles of the present disclosure, a system, for delivering, deploying, and retrieving an anchor with respect to anatomical tissue, includes an anchor having a distal end forming a distal anchor portion, a proximal end forming a proximal anchor portion, and a medial portion between the distal anchor portion and the proximal anchor portion, the anchor being shiftable between an elongated delivery configuration and a deployed configuration. Optionally, the system further includes a delivery device comprising a flexible tubular element defining a lumen therethrough, the lumen configured to retain the anchor in the elongated delivery configuration therein; and a retrieval device comprising an anchor-engaging element configured to engage the proximal anchor portion to proximally retract the anchor from the deployment site, and a tubular element defining a lumen therethrough configured to retain the anchor in the elongated delivery configuration.

Optionally, the proximal anchor portion has a free end extending away from the medial portion of the anchor to be graspable by the retrieval device to remove the anchor from the deployment site.

In accordance with various principles of the present disclosure, a method of deploying an anchor with respect to anatomical tissue, and subsequently removing the anchor from the anatomical tissue, includes delivering an anchor in an elongated delivery configuration to the anatomical tissue; deploying the anchor to allow the anchor to shift from the elongated delivery configuration to a deployed configuration in which at least a distal anchor portion is formed extending transverse to and away from a medial portion of the anchor; and removing the anchor after a selected period of time by withdrawing the anchor proximally to cause the distal anchor portion to return to the elongated delivery configuration and be withdrawn proximally through the anatomical tissue and withdrawn therefrom.

Optionally the method further includes delivering the anchor in a delivery device defining a lumen configured to retain the anchor in the elongated delivery configuration, the delivery device having a tissue-penetrating distal end configured to penetrate through the anatomical tissue to deliver the distal end of the anchor to a distal side of the anatomical tissue to expand transverse to a medial portion of the anchor extending through the anatomical tissue. Optionally, the method further includes withdrawing the delivery device proximally and/or advancing the anchor distally from the lumen of the delivery device to deploy the distal end of the anchor distal to the anatomical tissue.

Optionally, the method further includes grasping a proximal free end of the anchor extending away from the anatomical tissue with an anchor-engaging element of a retrieval device. Optionally, the method further includes engaging an anchor-engaging retrieval feature on the anchor-engaging element with a retrieval feature on the proximal portion of the anchor.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For the sake of simplicity, similar features among the illustrated implantable devices are referenced with similar reference numbers with leading numerals corresponding the figure number illustrating the embodiment, reference being made to the descriptions of such elements in FIG. 1 for the sake of brevity. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an elevational view of an example of an embodiment of an anchor formed in accordance with various aspects of the present disclosure and deployed across non-adherent anatomical structures.

FIG. 2 illustrates another example of an embodiment of an anchor formed in accordance with various principles of the present disclosure.

FIG. 3 illustrates another example of an embodiment of an anchor formed in accordance with various principles of the present disclosure.

FIG. 4 illustrates another example of an embodiment of an anchor formed in accordance with various principles of the present disclosure.

FIG. 5 illustrates another example of an embodiment of an anchor formed in accordance with various principles of the present disclosure.

FIG. 6 illustrates another example of an embodiment of an anchor formed in accordance with various principles of the present disclosure.

FIG. 7 illustrates another example of an embodiment of an anchor formed in accordance with various principles of the present disclosure.

FIG. 8A illustrates an elevational view of an example of an embodiment of an anchor formed in accordance with various principles of the present disclosure being delivered to a deployment site with a delivery device formed in accordance with various principles of the present disclosure.

FIG. 8B illustrates an elevational view of an example of an embodiment of an anchor formed in accordance with various principles of the present disclosure being delivered and deployed with respect to a deployment site with a delivery device formed in accordance with various principles of the present disclosure.

FIG. 8C illustrates an elevational view of an example of an embodiment of an anchor formed in accordance with various principles of the present disclosure being deployed with respect to a deployment site with a delivery device formed in accordance with various principles of the present disclosure.

FIG. 9 illustrates an elevational view of an example of an embodiment of an anchor formed in accordance with various principles of the present disclosure being removed from a deployment site with a retrieval device formed in accordance with various principles of the present disclosure.

FIG. 10A illustrates an example of an embodiment of a retrieval device, formed in accordance with various principles of the present disclosure, being positioned to engage an example of an embodiment of an anchor-engaging element thereof with a retrieval feature of an example of an embodiment of an anchor formed in accordance with various principles of the present disclosure.

FIG. 10B illustrates a view similar to that of FIG. 10A, but with the anchor-engaging element engaged with the retrieval feature.

FIG. 11A illustrates another example of an embodiment of a retrieval device, formed in accordance with various principles of the present disclosure, being positioned to engage an anchor-engaging element thereof with a retrieval feature of another example of an embodiment of an anchor formed in accordance with various principles of the present disclosure.

FIG. 11B illustrates a view similar to that of FIG. 11A, but with the anchor-engaging element engaged with the retrieval feature.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, etc.) such location or site.

The present disclosure relates to generally low-profile medical devices which may be implanted or affixed with respect to anatomical tissue. For instance, a low-profile device formed in accordance with various principles of the present disclosure is sized, shaped, configured, and/or dimensioned to be delivered transluminally through a patient's body, such as through a flexible tubular element guided within the patient to a deployment site for the device. In some embodiments, the low-profile device is formed from a flexible wire delivered within the lumen of a flexible tubular element. It will be appreciated that terms such as implant or affix (and other grammatical forms thereof) may be used interchangeably herein with terms (and grammatical forms thereof) such as anchor, attach, associate, couple, engage, embed, hold, retain, purchase, secure, etc., without intent to limit. It will further be appreciated that the term anchor (and other grammatical forms thereof) is used for the sake of convenience and may be used interchangeably herein with terms (and other grammatical forms thereof) such as anchor component, anchor device, anchor element, anchor mechanism, anchoring component, anchoring device, anchoring element, anchoring mechanism, and the like, such terms being known in the art to represent structures configured to hold another object in place. For the sake of convenience, and without intent to limit, reference is made herein to an anchor or a device interchangeably without intent to limit. An example of a use of such anchor is to hold anatomical tissues, such as tissue walls of non-adherent anatomical structures, in apposition with each other, although the use of devices, systems, and methods disclosed herein are not limited in this manner, and have other and/or broader uses and applications. For instance, although the accompanying drawings illustrate an anchor deployed across non-adherent anatomical structures, the anchor may instead be deployed with respect to adjacent tissue walls of the same anatomical structure, such as to close an opening in such anatomical wall. Additionally or alternatively, an anchor formed in accordance with various principles of the present disclosure may be used to hold together or to affix other devices or anatomical structures with respect to each other.

Devices and systems formed in accordance with various principles of the present disclosure may be delivered in a generally elongated manner to facilitate delivery within and through a patient's body (e.g., transluminally such as endoscopically, transcatheterally, etc.). A pusher may be provided within the delivery device to distally advance the anchor out of a distal open end of the delivery device. Additionally or alternatively, the delivery device may be proximally withdrawn with respect to the anchor. In some embodiments, an anchor is delivered to a deployment site in an elongated configuration within an appropriate delivery needle. A distal end of the device is extended through anatomical tissue to deploy the device (and, if a delivery needle is used, the distal end of such delivery needle is extended through the anatomical tissue). Upon deployment, the device shifts to a deployed configuration with anchor portions at one or both ends thereof. The anchor portions may be curved or otherwise extend transverse to a medial portion of the device which may remain elongated or may otherwise bend or deform in a manner suitable to the deployment site and/or use thereof as determined by the medical professional utilizing such device. At least a portion of the medial portion may extend through tissue with respect to which the anchor is deployed. In some embodiments, the devices are formed of a biocompatible material having sufficient elasticity so that the devices transition upon deployment from the delivery configuration to the deployed configuration. Examples of materials include, without limitation, nitinol or another biocompatible elastic or superelastic material, including, without limitation, plastics such as high performance polyethyenes (HPPE), low density polyethylenes (LDPE), Nylons, polyetheretherketone (PEEK), Polyetherimide (PEI), etc., or other materials with sufficient elasticity, stiffness, and/or ductility capable of delivery through narrow passages yet shifting to a configuration capable of being held with respect to tissue, In some embodiments, at least the distal end shifts into a distal anchor portion enlarged and/or transverse to the medial portion. For instance, the distal anchor portion may be a curved configuration presenting an enlarged anchor portion at the distal end of the anchor. Such configuration inhibits inadvertent proximal withdrawal of the anchor and/or shifting of the tissue with respect to the anchor portion. Upon securing the proximal end of the anchor with respect to the tissue (e.g., through which the anchor is inserted/implanted), the anchor may be used to hold adjacent non-adherent anatomical structures position (e.g., in apposition) with respect to each other. In some embodiments, the proximal end of the anchor is secured by shifting the proximal end into a proximal anchor portion enlarged and/or transverse to a medial portion of the anchor, although other manners of securement are within the scope and spirit of the present disclosure.

In accordance with various principles of the present disclosure, a system includes a delivery device with a lumen extending therethrough. The lumen of such delivery device is configured for a device formed in accordance with various principles of the present disclosure to be delivered therein to a deployment site. The device may be maintained in the elongated delivery configuration within the lumen of the delivery device of the associated system. Once the device is deployed from the lumen of the delivery device, the device extends into the deployed configuration. For instance, if the device is formed of an clastic (optionally shape memory) material, the device may automatically shift to the deployed configuration upon deployment without further action upon the device. As may be appreciated, the lumen of the delivery device may retain the device in its delivery configuration. In some embodiments, the delivery device includes a flexible tubular element having a sufficiently small diameter to maintain a low profile of the device as understood by those of ordinary skill in the art with reference to the deployment site and/or procedure to be performed. For instance, the flexible tubular element may be a 19 gauge needle (e.g., a fine needle aspiration (FNA) needle or a fine needle biopsy (FNB) needle) in which the anchor is delivered, and which may also facilitate delivery of the anchor. As may be appreciated by those of ordinary skill in the art, a 19 gauge needle has a generally accepted low profile which may puncture tissue to deploy a device formed in accordance with various principles of the present disclosure securely through tissue without leaving excess space between the device and the tissue. Use of a low profile flexible tubular element to deliver the anchor and optionally also to deploy the anchor into tissue thereby reduces if not eliminates the risk of leakage through the tissue and/or ensures a tight fit of the device with respect to the tissue and/or allows for ready healing of the puncture and/or quick closure and healing if the device is later removed from the tissue.

An anchor formed in accordance with various principles of the present disclosure may be configured to hold non-adherent anatomical structures together temporarily during a procedure. Additionally or alternatively, once an anchor formed in accordance with various principles of the present disclosure has been deployed, another device may be deployed over or adjacent the anchor. For instance, a stent may be deployed across non-adherent anatomical structures held in apposition by an anchor formed in accordance with various principles of the present disclosure, either over the anchor or adjacent the anchor. The stent may form an anastomosis across the non-adherent anatomical structures. For example, the non-adherent anatomical structures may be a stomach and a jejunum, the anchor formed in accordance with various principles of the present disclosure holding the stomach and jejunum in apposition for a stent to be deployed across the stomach and jejunum to form a gastrojejunostomy. The anchor may be deployed to hold non-adherent anatomical structures in apposition to prevent leakage of materials through a stent extended across such non-adherent anatomical structures until an anastomosis is formed. Once an anastomosis has been formed, or sufficient tissue ingrowth has occurred to cause the non-adherent anatomical structure to be adhered to each other, the anchor may be removed from the deployment site.

In some embodiments, an anchor formed in accordance with various principles of the present disclosure may be removable from its deployment site with respect to tissue, such as after a procedure facilitated by the anchor has been completed. More particularly, the devices and systems may facilitate removal in one or more manners. In some embodiments, the proximal end of the device is configured to be accessible by a removal device. For instance, even if the proximal end of the anchor shifts into an anchoring configuration (e.g., forms a proximal anchor portion), a portion thereof, such as a free end of the anchor, remains accessible for grasping by a removal device. The removal device may be a flexible tubular element with a lumen defined therein, and configured to proximally withdraw the anchor into the lumen. The removal device may return the anchor to the delivery configuration (or a configuration close to the delivery configuration to allow and/or facilitate removal in a similar manner in which the anchor has been delivered). The distal anchor portion is likewise configured to return to the delivery configuration to permit proximal withdrawal of the anchor, withdrawing the distal anchor portion proximally through the deployment site and into the delivery device.

Various embodiments of devices, systems, and methods for anchoring to tissue will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments are generally designated with the same reference numbers (under 1000) increased by a multiple of 100 and redundant description is generally omitted for the sake of brevity. Moreover, certain features in one embodiment may be used across different embodiments and are not necessarily individually labeled when appearing in different embodiments.

Turning now to the drawings, an example of an embodiment of an anchor 100 formed in accordance with various principles of the present disclosure is illustrated deployed with respect to and extending across a proximal non-adherent anatomical structure P and a distal non-adherent anatomical structure D. Although the proximal non-adherent anatomical structure P and a distal non-adherent anatomical structure D are illustrated spaced apart from each other, it will be appreciated that the anchor 100 may hold these structures in apposition with respect to each other. For instance, the proximal non-adherent anatomical structure P may be a patient's stomach and the distal non-adherent anatomical structure D may be a portion of the patient's jejunum, and the anchor 100 may be used to facilitate formation of an anastomosis (gastrojejunostomy) therebetween. Although not illustrated, the anchor 100 may be extended across a single tissue wall, or across more than one tissue wall of the same anatomical structure (e.g., tissue walls on either side of a lesion, cut, etc., in the anatomical structure, joined together by an anchor 100 formed in accordance with various principles of the present disclosure to treat or repair the lesion, cut, etc.), or otherwise, the present disclosure not being limited in this manner.

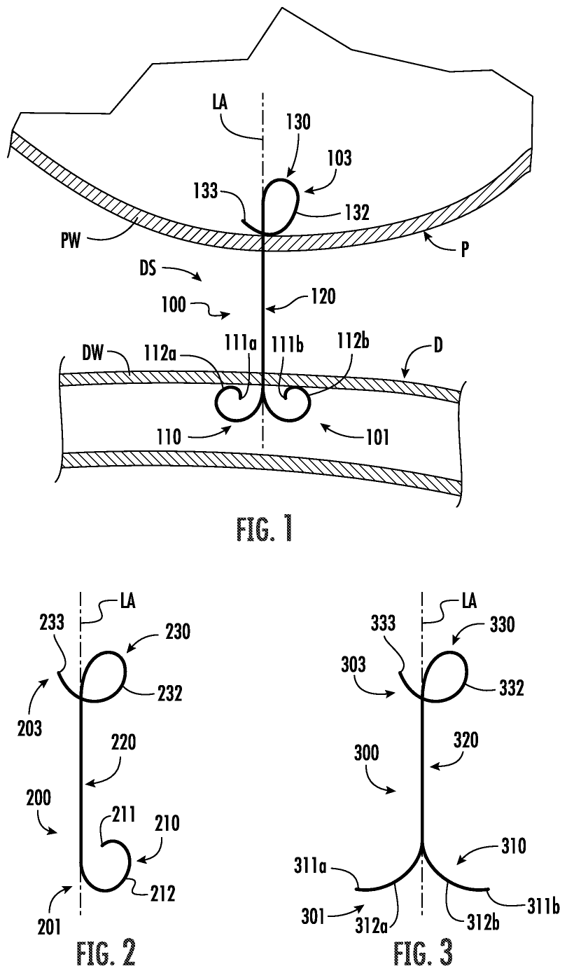

In the example of an embodiment illustrated in FIG. 1, the anchor 100 is generally elongated and has a distal end 101 and a proximal end 103. In the illustrated deployed configuration, a portion of the anchor 100 along the distal end 101 thereof forms/is formed into a distal anchor portion 110 configured to hold the anchor 100 with respect to a deployment site DS. For instance, the distal anchor portion 110 extends outwardly transverse to a medial portion 120 of the anchor 100 to resist proximal withdrawal from the deployment site DS (in the direction from which the anchor 100 is delivered) and/or distal movement of the tissue wall DW with respect to the distal anchor portion 110 of the anchor 120. In the illustrated example of an embodiment of a deployment site DS, the distal anchor portion 110 is anchored with respect to and distal to a tissue wall DW of the illustrated distal non-adherent anatomical structure D.

The medial portion 120 of the anchor 100 extends proximally through the tissue wall with respect to which the distal anchor portion 110 is positioned, the distal anchor portion 110 remaining at a distal side of the tissue wall DW. In embodiments in which a distal non-adherent anatomical structure DS is held in apposition with respect to a proximal non-adherent anatomical structure PS, the medial portion 120 extends through a tissue wall DW of the distal non-adherent anatomical structure DS as well as through a tissue wall PW of the proximal non-adherent anatomical structure PS. The proximal end 103 of the anchor 100 may be secured at the proximal side of the tissue wall through which the anchor 100 extends to hold the anchor 100 with respect to the deployment site DS and/or to hold the non-adherent anatomical structures P and D with respect to each other. The proximal end 103 of the anchor 100 may be secured with respect to the deployment site DS in a variety of manners, such as by being held by the medical professional, and/or by application of a separate anchor element with respect thereto (e.g., a clip, an adhesive bead, or other enlarged element anchoring the proximal end 103 of the anchor 100 with respect to the deployment site DS) and/or by being deformed into an anchor configuration (e.g., a knot or bend or other configuration generally transverse to the medial portion 120) to inhibit movement thereof with respect to the tissue through which the anchor 100 is deployed and/or to hold the distal non-adherent anatomical structure DS in place with respect to the proximal non-adherent anatomical structure PS. In some embodiments, the proximal end 103 may form/be formed into a proximal anchor portion 130 extending outwardly transverse to the medial portion 120 of the anchor 100, similar to the distal anchor portion 110 described above, respective configurations of such anchor portions being described in further detail below.

An anchor 100 such as described herein may be formed of an elastic/superelastic, optionally shape memory, material configured to shift a distal portion (adjacent the distal end 101), and optionally also a proximal portion (adjacent the proximal end 103), of the anchor 100 into an anchor configuration upon deployment at a deployment site. For instance, the anchor 100 may be formed such that one or both ends 101, 103 shift into an anchor portion 110, 130 extending transverse to the longitudinal axis LA of the elongated anchor 100 along which the medial portion 120 of the anchor 100 generally extends. In accordance with various further principles of the present disclosure, the flexibility of the material of the anchor 100 allows the anchor portion 110, 130 to return to an elongated configuration upon application of a retrieval force on the anchor 100. The anchor 100 may thereby be delivered in a generally compact delivery configuration, and optionally retrieved in a generally compact configuration, such as in a manner generally opposite the manner in which the anchor 100 is deployed. Various configurations of distal portions and proximal portions of anchors configured to hold an anchor formed in accordance with various principles of the present disclosure in place with respect to its deployment site, and optionally also to hold two non-adherent anatomical structure with respect to each other (e.g., in apposition), are illustrated in FIGS. 1-7.

In the example of an embodiment of an anchor 100 illustrated in FIG. 1, the distal anchor portion 110 curves/is curved away from (e.g., transverse to) the longitudinal axis LA of the anchor 100 into two curved portions 112a, 112b. In some embodiments, the element from which the anchor 100 is formed is cut longitudinally to form the curved portions 112a, 112b. The curved portions 112a, 112b branch away from each other to form a ram horn configuration. Respective free ends 111a, 111b of the curved portions 112a, 112b may be parallel to and/or directed away from the tissue wall DW of the distal non-adherent anatomical structure D. For instance, as illustrated in FIG. 1, the free ends 111a, 111b of the distal anchor portion 110 extend away from the tissue wall DW. As may be appreciated, the illustrated configuration of a distal anchor portion 110 allows the distal end 101 of the anchor 100 to be readily reconfigured from an elongated delivery configuration into the distal anchor portion 110 (e.g., by “blooming” into such configuration, such as in contrast with snapping into an anchor configuration) capable of retaining the anchor 100 with respect to the deployment site DS. Moreover, upon application of sufficient proximal force to the anchor 100, the distal anchor portion 110 may return to the elongated delivery configuration for retrieval, as described in further detail below.

As noted above, the proximal end 103 of the anchor 100 may also be configured to form a proximal portion 130 configured to hold the anchor 100 with respect to the deployment site. The proximal end 103 of the example of an embodiment of an anchor 100 illustrated in FIG. 1 curves/is curved away from the longitudinal axis LA of the anchor 100 into a proximal anchor portion 130 in the shape of a simple loop 132. Such configuration allows the proximal end 103 of the anchor 100 to be readily reconfigured from an elongated delivery configuration into a proximal anchor portion 130 upon deployment, and also allows the proximal anchor portion 130 to be returned to the elongated delivery configuration for retrieval, upon application of sufficient proximal force, as described in further detail below.

The example of an embodiment of an anchor 200 illustrated in FIG. 2 is similar to the example of an embodiment of an anchor 100 illustrated in FIG. 1, except the distal anchor portion 210 includes only one curved portion 212 extending transverse to the longitudinal axis LA. Such configuration may facilitate retrieval more readily than if two curved portions are formed at the distal end 201 of the anchor 200. Such configuration may also be easier to coat (e.g., with an electrically insulative material) than other configurations. Remaining elements of the example of an embodiment of an anchor 200 illustrated in FIG. 2 may be similar to the example of an embodiment of an anchor 100 illustrated in FIG. 1, and are labeled with similar reference numbers increased by 100, reference being made to the above descriptions of such elements for the sake of brevity.

The curved portions 112a, 112b of the distal anchor portion 110 of the anchor 100 illustrated in FIG. 1, and the curved portion 212 of the distal anchor portion 110 of the anchor 200 illustrated in FIG. 2 may be considered to curl back on themselves, with the respective free ends 111a, 111b, 211 thereof directed generally towards the distal end 101, 201 of the anchor 100, 200. However, in some embodiments, as illustrated in FIG. 3, an anchor 300 may have a distal anchor portion 310 with curved portions 312a, 312b curving away from (e.g., transverse to) the longitudinal axis LA of the anchor 100 yet not curling back on themselves. In such embodiment, it may be desirable for the respective free ends 311a, 312b of the curved portions 312a, 312b to be substantially parallel to the tissue wall through which the anchor 300 extends. Alternatively, the respective free ends 311a, 311b of the curved portions 312a, 312b of the distal anchor portion 310 may extend into the tissue wall through which the anchor 300 extends, such as to increase the anchoring force of the distal anchor portion 310 with respect to the deployment site. It will be appreciated that the material forming the anchor 300 illustrated in FIG. 3 may be somewhat stiffer than the material forming the anchor 100 illustrated in FIG. 1 or the anchor 200 illustrated in FIG. 2 (which anchors have increased curvature and optionally also length) so that the curved portions 312a, 312b may achieve a sufficient hold with respect to the deployment site DS. It will be appreciated that other elements of the example of an embodiment of an anchor 300 illustrated in FIG. 3 may be similar to the example of an embodiment of an anchor 100 illustrated in FIG. 1, and are labeled with similar reference numbers increased by 200, reference being made to the above descriptions of such elements for the sake of brevity.

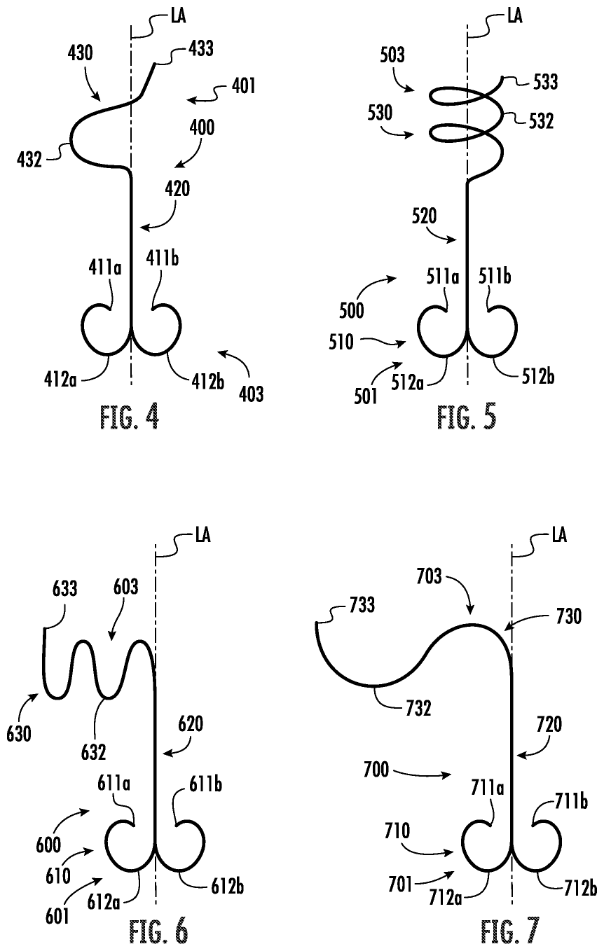

Instead of an anchor 100 having a proximal anchor portion 130 formed as a simple loop 132, as illustrated in FIG. 1, the proximal anchor portion may have various other configurations, examples of which are illustrated in FIG. 4, FIG. 5, FIG. 6, and FIG. 7.

For instance, the example of an embodiment of an anchor 400 illustrated in FIG. 4 has a simplified proximal anchor portion 430 to facilitate retrieval of the anchor 400. More particularly, instead of forming a generally closed loop 132, as in the example of an embodiment illustrated in FIG. 1, the proximal anchor portion 430 includes a curved portion 432 extending transverse to the longitudinal axis LA of the anchor 400 (e.g., transverse to the medial portion 420) sufficiently to hold the proximal anchor portion 430 and the tissue at the deployment site with respect to each other. As a generally open curve, the curved portion 432 may be more readily returned to an elongated delivery configuration for retrieval (as described in further detail below) than the more closed loop 132, 232, 332 illustrated in FIG. 1, FIG. 2, and FIG. 3, respectively. It will be appreciated that the distal anchor portion 410 of the example of an embodiment of an anchor 400 illustrated in FIG. 4 is substantially the same as the distal anchor portion 110 of the example of an embodiment of an anchor 100 illustrated in FIG. 1. However, other configurations of a distal anchor portion 410 (such as described above or otherwise) are within the scope and spirit of the present disclosure, the configuration of the proximal anchor portion 430 not limiting the configuration of the distal anchor portion 410. It will be appreciated that other elements of the example of an embodiment of an anchor 400 illustrated in FIG. 4 may be similar to the example of an embodiment of an anchor 100 illustrated in FIG. 1, and are labeled with similar reference numbers increased by 300, reference being made to the above descriptions of such elements for the sake of brevity.

Instead of curling in generally a single plane, as in the examples of embodiments of anchors 100, 400 illustrated in FIG. 1 and FIG. 4, the proximal anchor portion 530 may be curled into a coil spring configuration about an axis generally parallel to longitudinal axis LA of the medial portion 520 of the anchor 500 with coils 532 generally transverse to the longitudinal extent of the medial portion 520, as illustrated in FIG. 5. Such configuration may firmly hold the anchor 500 with respect to the deployment site, yet allow for retrieval of the anchor 500, such as in a manner described below. It will be appreciated that other elements of the example of an embodiment of an anchor 500 illustrated in FIG. 5 may be similar to the example of an embodiment of an anchor 100 illustrated in FIG. 1, and are labeled with similar reference numbers increased by 400, reference being made to the above descriptions of such elements for the sake of brevity. However, it will also be appreciated that other configurations of a distal anchor portion 510 (such as described above or otherwise) are within the scope and spirit of the present disclosure, the configuration of the proximal anchor portion 530 not limiting the configuration of the distal anchor portion 510.

Similar to the example of an embodiment of an anchor 500 illustrated in FIG. 5, the examples of embodiments of anchors 600, 700 illustrated in FIG. 6 and FIG. 7 have proximal anchor portion 630, 730 with more than one curved portion 632, 732. However, unlike the coils 523 of the proximal anchor portion 530 of the example of an embodiment anchor 500 illustrated in FIG. 5, the curved portion 632, 732 of the examples of embodiments of anchors 600, 700 illustrated in FIG. 6 and FIG. 7 do not lie in a plane transverse to the longitudinal extent of the medial portion 620, 720 of the anchor 600, 700.

More particularly, in the example of an embodiment of an anchor 600 illustrated n FIG. 6, the proximal end 603 curves or is curved into a proximal anchor portion 630 with a sinusoidal shaped curved portion 632 extending in a direction away from and generally transverse to the medial portion 620. The sine waves (one, two, three, four, etc., waves) of the curved portion 632, and/or segments thereof, may be in the same plane or in more than one plane, such as in different respective planes which are transverse to one another. In some embodiments, the curved portion 632 curves about or around a longitudinal axis LA generally parallel to the medial portion 620 of the anchor 600. Moreover, the plane in which the curved portion 632 extends may be in the same plane, or in a plane transverse to the plane in which the curved portions 612a, 612b of the distal anchor portion 610 of the anchor 600 extends.

The shape of the proximal anchor portion 730 of the example of an embodiment of an anchor 700 illustrated in FIG. 7 is similar to the proximal anchor portion 630 of the example of an embodiment of an anchor 600 illustrated in FIG. 6, but further simplified. In particular, the proximal end 703 of the example of an embodiment of an anchor 700 illustrated in FIG. 7 curves or is curved into a proximal anchor portion 730 with an S-curve portion 732 (e.g., a simple, single sine wave). It will be appreciated that the curves of the S-curve portion 732 need not be coplanar, and the S-curve itself need not lie in a single plane. In some embodiments, the S-curve portion 732 curves about or around an axis generally parallel to the medial portion 720 of the anchor 700. Moreover, the plane in which the S-curve portion 732 of the anchors 700 lies may be in the same plane or in a plane transverse to the plane in which the curved portions 712a, 712b of the distal anchor portions 710 extends.

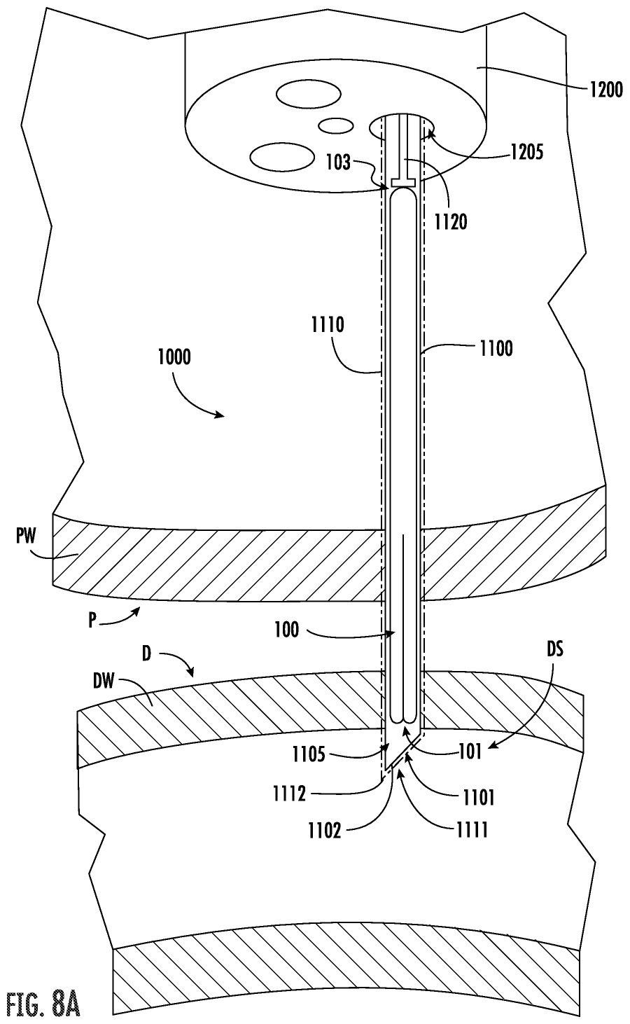

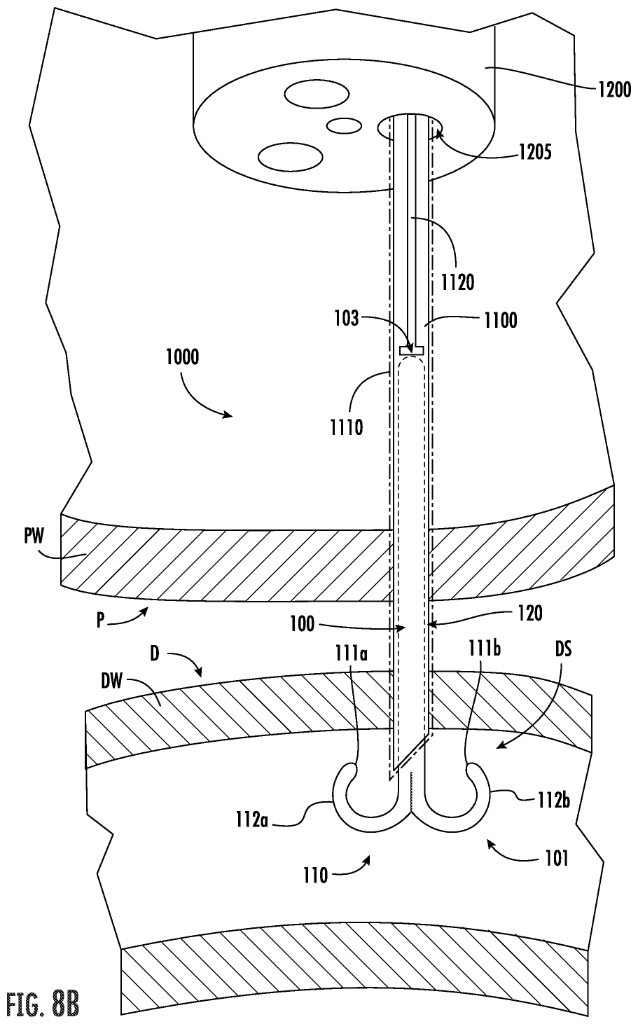

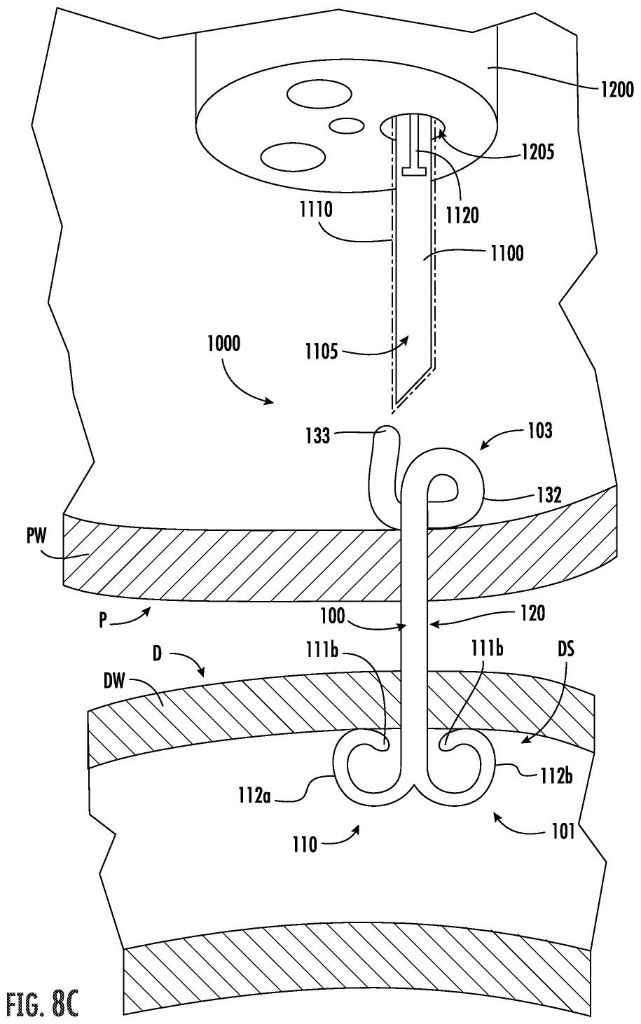

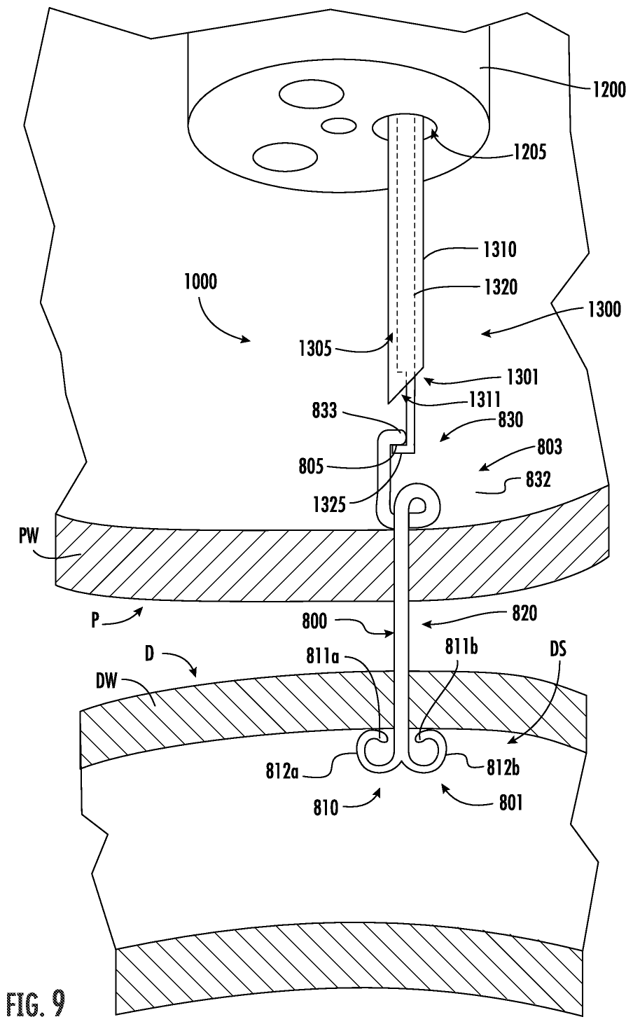

Any of the above-described anchors 100, 200, 300, 400, 500, 600, 700 may be formed in accordance with various principles of the present disclosure to be delivered transluminally (e.g., transcatheterally, endoscopically, etc.). To facilitate such delivery, the anchors 100, 200, 300, 400, 500, 600, 700 may be delivered in a generally compact, generally elongated configuration (with all or most portions or sections thereof extending along a longitudinal axis LA of the anchor). If the anchors 100, 200, 300, 400, 500, 600, 700 are formed from a shape memory material configured to form one or both of the anchor portions 110, 130, 210, 230, 310, 330, 410, 430, 510, 530, 610, 630, 710, 730 as described above, the anchors 100, 200, 300, 400, 500, 600, 700 may be delivered within a lumen of a flexible tubular delivery device configured to retain the anchors 100, 200, 300, 400, 500, 600, 700 in the delivery configuration. Any of the above-described anchors 100, 200, 300, 400, 500, 600, 700 may be a part of or used with an anchor system 1000 including elements facilitating delivery and/or deployment and/or retrieval of the anchor 100. An example of an embodiment of an anchor system 1000 is illustrated in FIGS. 8A-8C delivering an example of an embodiment of an anchor 100 formed in accordance with various principles of the present disclosure. An example of an embodiment of an anchor system 1000 is illustrated in FIG. 9 retrieving (proximally retracting and withdrawing) an example of an embodiment of an anchor 100′ similar to the anchor 100 illustrated in FIG. 1 but modified to facilitate retrieval.

More particularly, the example of an embodiment of anchor system 1000 illustrated in FIGS. 8A-8C includes a flexible tubular delivery device 1100 having a lumen 1115 defined therein. As illustrated in FIG. 8A, the lumen 1105 of the delivery device 1100 is sized, shaped, configured, and/or dimensioned for an anchor 100 formed in accordance with various principles of the present disclosure to fit within the lumen 1105 in a generally elongated delivery configuration for delivery to a deployment site. For instance, the anchor 100 may be formed of an elongate element, such as a wire, and the lumen 1105 may be dimensioned to retain such elongate element in an elongated configuration to facilitate delivery (e.g., transluminal delivery) of the anchor 100 within a patient's body and to a deployment site DS. It will be appreciated that although an anchor 100, such as illustrated in FIG. 1, is illustrated in FIGS. 8A-8C, any of the other above-described anchors 200, 300, 400, 500, 600, 700, or another anchor formed in accordance with various principles of the present disclosure, may be used with the anchor system 1000 described herein.

In some embodiments, the delivery device 1100 includes a tissue-penetrating end 1102 at a distal end 1101 of the delivery device 1100. The tissue-penetrating end 1102 is configured to penetrate tissue walls to deliver the distal end 101 of the anchor 100 to the deployment site DS. In some embodiments, the tissue-penetrating end 1102 may be a sharp end capable of puncturing tissue. Additionally or alternatively, a separate tissue-penetrating delivery device 1110 may form an entry site for the delivery device 1100 and anchor 100, and also deliver the delivery device 1100 and anchor 100 to the deployment site DS. Such optional tissue-penetrating delivery device 1110 (illustrated in phantom) includes a sharp tissue-penetrating end 1112 at a distal end 1111 of the tissue-penetrating delivery device 1110. The sharp tissue-penetrating end 1112 is capable of puncturing and penetrating anatomical tissue to deliver the delivery device 1100 and the anchor 100 (positioned within the lumen 1105 of the delivery device 1100) through anatomical tissue, as illustrated in FIG. 8A. In embodiments including a separate tissue-penetrating delivery device 1112, the distal end 1101 of the delivery device 1100 may be generally blunt and not include a tissue-penetrating end 1102. Optionally, the anchor system 1000 includes or is used with an endoscope 1200 having a working channel 1205 through which the delivery device 1100 (and the separate tissue-penetrating delivery device 1110, if included) and the anchor 100 are delivered to the deployment site DS.

In the example of an embodiment illustrated in FIG. 8A, FIG. 8B, and FIG. 8C, the example of an embodiment of an anchor 100 is illustrated as being deployed across adjacent non-adherent anatomical structures. The sharp tissue-penetrating end 1101 of the delivery device 1100 or the sharp tissue-penetrating end 1112 of the tissue-penetrating delivery device 1110 punctures through the proximal non-adherent anatomical structure PS and the distal non-adherent anatomical structure DS to access a deployment site distal to the distal non-adherent anatomical structure DS. The delivery device 1100 may then be proximally retracted, as illustrated in FIG. 8B, to deploy the distal end 101 of the anchor 100. Additionally or alternatively, a pusher 1120 may be provided within the delivery device 1100 and configured to engage the proximal end 103 of the anchor 100 to push the anchor 100 out of the delivery device 1100 and into the deployed configuration. As may be appreciated with reference to FIG. 8B, as the distal end 101 of the anchor 100 exits the lumen 1105 of the delivery device 1100, the distal end 101 shifts to form the distal anchor portion 110 configured to anchor with respect to tissue at the deployment site. The delivery device 1100 may then be further retracted, as illustrated in FIG. 8C, to deploy the proximal end 103 of the anchor 100 and allow the proximal anchor portion 130 to be formed, holding the anchor 100 in place with respect to the deployment site, holding the tissue wall PW of the proximal non-adherent anatomical structure PS in place with respect to the tissue wall DW of the distal non-adherent anatomical structure DS.

In accordance with various principles of the present disclosure, any or all of the anchors 100, 200, 300, 400, 500, 600, 700 described above may be configured to facilitate retrieval from a deployment site. For instance, the proximal free ends 133, 233, 333, 433, 533, 633, 733 of the examples of embodiments of anchors 100, 200, 300, 400, 500, 600, 700 illustrated in FIG. 1, FIG. 2, FIG. 3, FIG. 4, FIG. 5, FIG. 6, and FIG. 7 face away from the deployment site and towards the direction from which the anchor has been delivered. The orientation and/or configuration of the proximal free ends 133, 233, 333, 433, 533, 633, 733 may facilitate accessibility thereof, such as to access and proximally withdraw the anchor 100, 200, 300, 400, 500, 600, 700 from the deployment site by a retrieval device. The retrieval device may be in any desired form known or heretofore known by those of ordinary skill in the art configured to grasp an end of a device, such as a free end 133, 233, 333, 433, 533, 633, 733 of any of the examples of embodiments of anchors 100, 200, 300, 400, 500, 600, 700 described herein and illustrated in FIG. 1, FIG. 2, FIG. 3, FIG. 4, FIG. 5, FIG. 6, and FIG. 7, respectively.

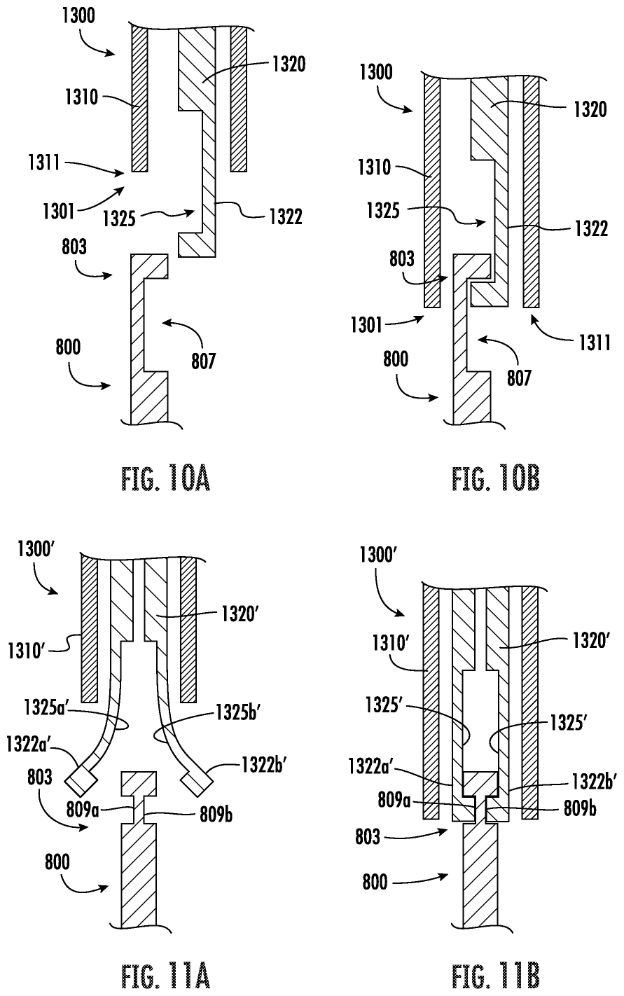

In some embodiments, such as illustrated in FIG. 9, FIG. 10A, FIG. 10B, FIG. 11A, and FIG. 11B, an anchor includes a retrieval feature facilitating engagement by a retrieval device for removal of the anchor from the deployment site by the retrieval device. It will be appreciated that although the anchor 800 illustrated in FIG. 9, FIG. 10A, FIG. 10B, FIG. 11A, and FIG. 11B appears similar to the anchor 100 illustrated in FIG. 1, the principles of retrieval of an anchor may be applied to anchors of any other configuration, such as, without limitation, the configuration of any of the anchors 200, 300, 400, 500, 600, 700 illustrated in FIG. 2, FIG. 3, FIG. 4, FIG. 5, FIG. 6, and FIG. 7. Furthermore, it will be appreciated that the illustrated anchor 800 is shown with a first embodiment of a retrieval feature 805 in FIG. 9, with a second embodiment of a retrieval feature 807 in FIG. 10A and FIG. 10B, and with a third embodiment of a retrieval feature 809 in FIG. 11A and FIG. 11B. It will be appreciated that features of the anchor 800 illustrated in FIG. 9, FIG. 10A, FIG. 10B, FIG. 11A, and FIG. 11B FIG. 10A and FIG. 10B labeled with the same reference numbers are substantially the same. Moreover, it will be appreciated that elements of the example of an embodiment of an anchor 800 illustrated in FIG. 9, FIG. 10A, FIG. 10B, FIG. 11A, and FIG. 11B which are similar to elements of the example of an embodiment of an anchor 100 illustrated in FIG. 1 are labeled with similar reference numbers increased by 700, reference being made to the above descriptions of such elements for the sake of brevity. The various devices, systems, and method described with reference to FIG. 9, FIG. 10A, FIG. 10B, FIG. 11A, and FIG. 11B may be applied to any of the examples of embodiments of retrieval features described herein, as will now be described.

An example of an embodiment of an anchor 800 formed with a retrieval feature 805 facilitating retrieval thereof from a deployment site DS is illustrated in FIG. 9. The illustrated example of an embodiment of an anchor 800 includes a retrieval feature 805 formed adjacent a free end 833 of the proximal anchor portion 830 formed at the proximal end 803 of the anchor 800. As also illustrated in FIG. 9, the anchor system 1000 may include a retrieval device 1300 configured to engage and to retrieve the anchor 800. I illustrated example of an embodiment of a retrieval device 1300 includes a flexible tubular element 1310 configured to deliver an anchor-engaging element 1320. In some embodiments, the flexible tubular element 1310 may be the same as or similar to the above-described delivery device 1100. The flexible tubular element 1310 may have a blunt end if the anchor-engaging element 1320 pulls the anchor 100 proximally into the flexible tubular element 1310. However, if the distal end of the flexible tubular element 1310 has a sharp tissue penetrating end, the outer diameter may be selected such that the flexible tubular element 1310 may extend through the deployment site and over the distal anchor portion 110 to shift the distal anchor portion 110 into a delivery configuration for retrieval from the deployment site. The anchor-engaging element 1320 includes an anchor-engaging portion 1322 having an anchor-engaging retrieval feature 1325 configured to mate with a retrieval feature along a proximal end 103 of an anchor 800. For instance, the anchor-engaging retrieval feature and the retrieval feature on the anchor may be mating notches or other interengaging shapes, such as the example of an embodiment of a retrieval feature 805 illustrated on the anchor 800, or the alternative embodiment of a retrieval feature 807 illustrated in FIG. 10A and FIG. 10B.

As illustrated in further detail in FIG. 10A, once the distal end 1301 of the retrieval device 1300 is near the proximal end 803 of the anchor 800, the flexible tubular element 1310 may be proximally retracted and/or the anchor-engaging element 1320 distally advanced to extend the anchor-engaging portion 1322 of the anchor-engaging element 1320 distally out of the distal end 1311 of the flexible tubular element 1310 and towards the proximal end 803′ of the anchor 800. The anchor-engaging retrieval feature 1325 of the anchor-engaging element 1320 may then be engaged with the retrieval feature 805 of the anchor 800 illustrated in FIG. 9, of the retrieval feature 807 illustrated in FIG. 10A and FIG. 10B. To hold the anchor-engaging retrieval feature 1325 on the anchor-engaging portion 1322 in engagement with the retrieval feature 805, 807 on the anchor 800, the flexible tubular element 1310 is advanced thereover, such as illustrated in FIG. 10B. Either the anchor-engaging element 1320 alone or with the flexible tubular element 1310 as well may then be proximally retracted to proximally withdraw the anchor 800 from the deployment site. As may be appreciated, the anchor 800 is returned to a generally elongated delivery configuration when retracted proximally into the flexible tubular element 1310 for retrieval from the patient's body.

Although the anchor-engaging element 1320 illustrated in FIG. 10A and FIG. 10B is illustrated with a single anchor-engaging retrieval feature 1325, it will be appreciated that other configurations are within the scope and spirit of the present disclosure. For instance, the example of an embodiment of a retrieval device 1300′ illustrated in FIG. 11A and FIG. 11B has an anchor-engaging element 1320′ with two or more anchor-engaging portions 1322′, 1322b′, each with an anchor-engaging retrieval features 1325′, 1325b′, respectively. Likewise, the example of an embodiment of an anchor 800 has two or more retrieval features 809a, 809b (e.g., a number corresponding to the number of anchor-engaging retrieval features). Provision of more than one anchor-engaging retrieval features 1325a, 1325b and more than one retrieval feature 809a, 809b may facilitate alignment of the anchor-engaging retrieval features 1325a′, 1325b′ with respective retrieval features 809a, 809b on the anchor 800 to facilitate capture of the anchor 800 by the anchor-engaging portion 1322 of the anchor-engaging element 1320. Once the anchor-engaging retrieval features 1325′, 1325b′ have engaged with a respective retrieval features 809a, 809b on the anchor 800, the flexible tubular element 1310 is advanced over the anchor-engaging portion 1322 and the proximal end 803 of the anchor 800 to retain the engagement of the anchor-engaging retrieval features 1325a′, 1325b′ and the retrieval features 809a, 809b. The anchor-engaging element 1320′, alone or with the flexible tubular element 1310, may then be proximally retracted to proximally withdraw the anchor 800 from the deployment site.

Various modifications may be made to the above-described embodiments without departing from the scope and spirit of the present disclosure. For instance, although the anchors described above have anchor portions formed from the same elongated element, it will be appreciated that the anchor portions may be separately formed (e.g., from a shape memory material, such as described above) and coupled together with a suture. Alternatively, the proximal anchor portion may be a suture coupled with a distal anchor portion formed from another material, such as a shape memory material. To release the anchor from the deployment site, the suture may simply be cut, allowing the distal anchor portion to pass out of the body naturally. Preferably, a biocompatible material is used, and the size of the distal anchor portion is sufficiently small as to not affect the patient when passing out of the patient. The proximal portion may be retrieved when cutting the suture to release the distal and proximal anchor portions to release the anchor from the deployment site.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

Although embodiments of the present disclosure may be described with specific reference to medical devices and systems and procedures applied to the gastrointestinal system, it should be appreciated that such medical devices, systems, and procedures may be used to treat tissues of the abdominal cavity, digestive system, urinary tract, reproductive tract, respiratory system, cardiovascular system, circulatory system, and the like. Various further benefits of the various aspects, features, components, and structures of devices, systems, and methods such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An anchor capable of extending through anatomical tissue at a deployment site, said anchor comprising:
a distal end forming a distal anchor portion;
a proximal end forming a proximal anchor portion; and
a medial portion, between the distal end and the proximal end, configured to extend through anatomical tissue;
wherein:
said anchor is shiftable between an elongated delivery configuration and a deployed configuration;
at least said distal anchor portion is formed when said anchor shifts into the deployed configuration by bending into a configuration extending transverse to said medial portion; and
a free end of said proximal anchor portion extends away from the deployment site to facilitate access thereto for removal of the anchor from the deployment site.

2. The anchor of claim 1, wherein at least one of said distal anchor portion or said proximal anchor portion is curved.

3. The anchor of claim 2, wherein said distal anchor portion is curved in a direction transverse to and away from said medial portion.

4. The anchor of claim 3, wherein said distal anchor portion has a free end directed away from said medial portion.

5. The anchor of claim 3, wherein said distal anchor portion has a free end extending along the anatomical tissue.

6. The anchor of claim 3, wherein said distal anchor portion has a free end embedded into the anatomical tissue.

7. The anchor of claim 3, wherein said proximal anchor portion is curved in a direction transverse to an away from said medial portion.

8. The anchor of claim 7, wherein said proximal anchor portion has a free end extending away from said medial portion to be graspable to remove said anchor from the deployment site.

9. The anchor of claim 8, wherein said anchor is shiftable from the deployed configuration to the delivery configuration upon being pulled into a lumen of a tubular element for removal from the deployment site.

10. The anchor of claim 1, wherein said anchor is formed from a shape memory material.

11. The anchor of claim 7, wherein said anchor is formed from a wire.

12. The anchor of claim 1, wherein said medial portion comprises a suture.

13. The anchor of claim 12, wherein said anchor is removable from the deployment site upon cutting the suture.

14. A system for delivering, deploying, and retrieving an anchor with respect to anatomical tissue, said system comprising:
an anchor having a distal end forming a distal anchor portion, a proximal end forming a proximal anchor portion, and a medial portion between the distal anchor portion and the proximal anchor portion, said anchor being shiftable between an elongated delivery configuration and a deployed configuration;
a delivery device comprising a flexible tubular element defining a lumen therethrough, the lumen configured to retain said anchor in the elongated delivery configuration therein; and
a retrieval device comprising an anchor-engaging element configured to engage said proximal anchor portion to proximally retract the anchor from the deployment site, and a tubular element defining a lumen therethrough configured to retain said anchor in the elongated delivery configuration.

15. The system of claim 14, wherein said proximal anchor portion has a free end extending away from said medial portion of said anchor to be graspable by said retrieval device to remove said anchor from the deployment site.

16. A method of deploying an anchor with respect to anatomical tissue, and subsequently removing the anchor from the anatomical tissue, said method comprising:
delivering an anchor in an elongated delivery configuration to the anatomical tissue;
deploying the anchor to allow the anchor to shift from the elongated delivery configuration to a deployed configuration in which at least a distal anchor portion is formed extending transverse to and away from a medial portion of the anchor; and
removing the anchor after a selected period of time by withdrawing the anchor proximally to cause the distal anchor portion to return to the elongated delivery configuration and be withdrawn proximally through the anatomical tissue and withdrawn therefrom.

17. The method of claim 16, further comprising delivering the anchor in a delivery device defining a lumen configured to retain the anchor in the elongated delivery configuration, the delivery device having a tissue-penetrating distal end configured to penetrate through the anatomical tissue to deliver the distal end of the anchor to a distal side of the anatomical tissue to expand transverse to a medial portion of the anchor extending through the anatomical tissue.

18. The method of claim 17, further comprising withdrawing the delivery device proximally and/or advancing the anchor distally from the lumen of the delivery device to deploy the distal end of the anchor distal to the anatomical tissue.

19. The method of claim 16, further comprising grasping a proximal free end of the anchor extending away from the anatomical tissue with an anchor-engaging element of a retrieval device.

20. The method of claim 19, further comprising engaging an anchor-engaging retrieval feature on the anchor-engaging element with a retrieval feature on the proximal portion of the anchor.
